(19) United States
(12) Patent Application Publication
(10) Pub. No.: US 2013/0190270 A1
(43) Pub. Date: Jul. 25, 2013

(54) Method for Improving Vein Performance in Bypass Surgery

(71) Applicant: Hans W. J. Niessen, Amsterdam (NL)

(72) Inventor: Hans W. J. Niessen, Amsterdam (NL)

(21) Appl. No.: 13/748,002

(22) Filed: Jan. 23, 2013

Related U.S. Application Data
(60) Provisional application No. 61/590,058, filed on Jan. 24, 2012.

(51) Int. Cl. A61K 31/231; A61K 31/727; A01N 1/02

(52) U.S. Cl. CPC A61K 31/231; A01N 1/0226; A61K 31/727
USPC 514/56; 514/547; 435/1.1

(57) ABSTRACT

Methods to reduce damage to organelles, cells, tissues, organs and organ systems or components thereof caused by strain due to mechanical stress, including stretch stress and shear stress, are provided. The methods involve treating the organelles, cells, tissues, organs, and organs systems or components thereof (such as veins used for grafts in bypass surgery) with PX-18 and related compounds. Treatment with PX-18 and related compounds reduces damage due to stress and improves the functioning of the cells, tissues, organs or organs systems or components thereof. For example, the methods prevent the buildup of atherosclerotic plaque in transplanted (grafted) veins after coronary bypass surgery.

### CROSS-REFERENCE TO RELATED APPLICATIONS

This application claims priority to U.S. Provisional Application 61/590,058, filed Jan. 24, 2012, and the complete contents thereof is herein incorporated by reference.

### DESCRIPTION

### Background of the Invention

1. Field of the Invention

The invention generally provides methods to reduce damage to organelles, cells, tissues, organs and organ systems or components thereof caused by strain due to stretch stress and shear stress. In particular, the invention provides methods of treating organelles, cells, tissues, organs, and organs systems or components thereof (such as veins used for grafts in bypass surgery) with PX-18 and related PX compounds. Treatment with PX-18 and related PX compounds reduces damage due to stress and improves the functioning of organelles, cells, tissues, organs and organ systems and/or components thereof. For example, the methods prevent the buildup of atherosclerotic plaque in transplanted (grafted) veins after coronary bypass surgery.

2. Background of the Invention

Coronary artery bypass grafting (CABG) developed in the 1960s, utilizing the saphenous vein graft as the gold standard for the treatment of multivessel coronary artery disease. Despite the superiority of arterial graft patency over that of vein grafts, the multivessel nature of coronary artery disease and ready availability of saphenous veins still results in their use in over 70% of CABG procedures [1]. However the success of this procedure is limited by poor long-term vein graft patency [2].

Surgical preparation of the saphenous vein on its own injures the blood vessel, and as such, contributes to the poor long-term patency [3]. In addition, the increased pressure from the venous grafts also distends the arterial system which can eventually also cause pathological changes such as mural thinning and endothelial damage [4-8] and contributes to damage caused by shear stress [31-34]. Kockx et al. demonstrated that soon after grafting, the vein wall is infiltrated by polymorphonuclear leucocytes (PMN). This massive migration of PMN through the venous wall occurred simultaneously with endothelial damage, which is indicative of local inflammation [9]. Endothelial dysfunction after acute infection or inflammation may be a transient risk factor for abnormal vascular behavior and thus graft failure, which might be amenable to pharmacological intervention [10].

Furthermore, serum secretory phospholipase A2, type II-A (sPLA₂-IIA) could directly promote endothelial dysfunction by its ability to promote generation of lysophosphatidylcholine, a known inhibitor of endothelial nitric oxide synthase (eNOS) [12], and it has been demonstrated that the expression of sPLA₂-IIA in vascular smooth muscle cells (VSMC) plays an essential role in the progression of atherosclerotic plaques [13].

The prior art has thus far failed to provide compositions or methods for decreasing or eliminating damage to vein grafts.

### SUMMARY OF THE INVENTION

The present invention provides methods for treating organelles, cells, tissues, organs and organ systems or components thereof to prevent, reduce or treat damage caused by various stresses. The methods involve exposing the organelles, cells, tissues, organs and organ systems or components thereof to one or more compounds of the PX-18 family of compounds (e.g. PX-18 and compounds in the PX family). In one embodiment, at least one compound of the PX-18 family of compounds is used to treat veins used in bypass surgery to prevent or reduce damage that otherwise occurs to the veins during the process of harvesting the vein, during the time between harvest and grafting, and after grafting into a recipient. Treatment of veins with PX-18 during one or more of these time intervals results in a decrease in sPLA₂-IIA activity in the epithelial cells in the lumen of the vein, a decrease in thinning of the epithelium, and a decrease in long term damage to the vein. In addition, the build-up of arteriosclerotic plaque is seen to be avoided or lessened. The grafted veins treated by the methods of the invention display superior function and fewer complications than veins treated in the current state of the art but without PX-18.

The invention provides methods of protecting organelles, cells, tissues, organs and organ systems or components thereof from the deleterious effects of stretching and sheer stress and subsequent strain. The methods each comprise the step of treating said organelles, cells, tissues, organs and organ systems or components thereof with one or more compounds in the PX family of compounds. In one embodiment, the component of an organ system is a vein such as a saphenous vein. In another one embodiment, the vein is treated in vivo prior to harvesting said vein from a donor. In another embodiment, the vein is treated ex vivo after harvesting. In yet another embodiment, the vein is treated in vivo after it has been grafted into a recipient.

In an exemplary embodiment, a method of limiting endothelial damage in vein grafts and reducing the build-up of arteriosclerotic plaque in the vein grafts comprises providing one or more PX-18 or PX-18 related compounds to the endothelium of the vein used for the graft. In one embodiment, the step of providing is carried out by administering one or more PX-18 or PX-18 related compounds to a vein graft donor prior to removal of said vein graft from the donor. In another embodiment, the step of providing is carried out by ex vivo exposure of said vein graft to one or more PX-18 or PX-18 related compounds during storage, transport, or while the recipient is awaiting surgery, (i.e. after removal from the donor but prior to transplant into the recipient.) In another embodiment, the step of treatment is carried out by administering the one or more PX-18 or PX-18 related compounds to a vein graft recipient after the vein graft is grafted into said vein graft recipient. In another embodiment, the one or more PX-18 or PX-18 related compounds is provided to the vein graft recipient during an acute post-operative period, e.g. immediately after surgery and for a period of days or weeks after surgery. In another embodiment, the one or more PX-18 or PX-18 related compounds is provided to the vein graft recipient as long-term maintenance therapy, i.e. on an ongoing basis for months or years after the vein graft procedure.

A further embodiment of the invention is a method of limiting endothelial damage in vein grafts and reducing the build-up of arteriosclerotic plaque in vein grafts. The method comprises the step of providing a combination of an anticoagulant, such as heparin, and one or more PX-18 compounds to endothelium of said vein graft.

### BRIEF DESCRIPTION OF THE DRAWINGS

FIG. 1. Schematic representation of the in-vitro perfusion model.

FIGS. 2A and B. Immunohystochemical staining of perfused veins. (A) CD34 molecule positive area of intima of perfused veins with and without the sPLA2-IIA inhibitor PX-18. CD34 positive staining of microscopic sections of 6h perfused veins without (n=6) and with (n=6) PX-18. (B) 4A1 Immunohistochemical staining in a vein perfused for 6 h with the sPLA2-IIA inhibitor. In perfusion veins, focal weak sPLA2-IIA positivity was found in the VSMC, the longitudinal and circular layer of media, but not in (residual) endothelial cells. Arrows: sPLA2-IIA positivity. Magnification 400×.

FIGS. 3A and B. Effect of PX-18 on Human umbilical vein endothelial cells (HUVEC's) (A) Effect of PX-18 on caspase-3 activation in HUVECs subjected to mechanical strain. HUVECs were subjected to 24 hours of mechanical stretching with or without sPLA2-IIA inhibitor PX-18. Caspase-3 activity significantly decreased in the HUVECs with sPLA2-IIA inhibitor PX-18, compared with control HUVECs. Data represent mean±SEM.

(B) sPLA2 activity in HUVEC's. The PX-18 treated group of HUVEC's did not differ significantly from the non treated group. Negative: negative control namely the substrate containing assay mix without added lysate. Positive: positive control containing purified type III sPLA2; Control: stretched HUVECs without PX-18; PX-18: perfused HUVEC's with PX-18.

FIG. 4. Chemical structure drawing of PX-18.

### DETAILED DESCRIPTION

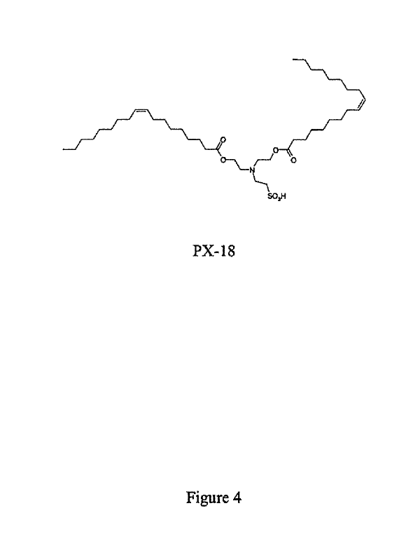

The PX family of compounds includes PX-18 (see FIG. 4) and other compounds satisfying Formula 1 below, and may be referred to as “PX-18 and related compounds” or “PX compound family” or “PX compounds”, etc. herein. The PX compounds are pharmacologically active, antioxidant, anti-phospholipase compounds that are chemically defined. In particular, each compound has at least two fatty moieties and no active hydroxy group. The compound may also have at least one ionizable group, which may be a carboxyl group, and each of the fatty moieties has from sixteen to twenty carbon atoms and at least one cis-unsaturated double bond. Members of the PX family of compounds are described in U.S. Pat. Nos. 6,600,059; 6,423,855; 6,020,489; 6,020,510; 5,859,271; and 5,659,055 (all to Franson, et al.), the complete contents of each of which is herein incorporated by reference.

Formula 1 shows a generic representation of the PX compound family which can be used in the practice of the invention,

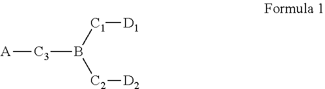

where A comprises H, OH, a sugar moiety, an ether, an ester, an amide, NH₂ or an acid or salt thereof; B is selected from the group consisting of N, NR, P, P═O, CH, and CR, wherein R is an alkyl chain of 1 to 6 carbons and the chain may be functionalized or non-functionalized; C₁, C₂ and C3 are connecting groups which may be the same or different and are selected from the group consisting —(CH₂)ₙ— and (CH₂CH₂—O)_{y} wherein n is an integer from 1 to 24, —(CH₂)ₙ— may be functionalized or non-functionalized, and y is an integer from 1 to 12; and D₁ and D₂ are fatty acid chains which may be different and are selected from the group consisting of fatty acid esters of the form CH₃ (CH₂)ₙCOO, and fatty acid amides of the form CH₃(CH₂)ₙCONH, wherein n is an integer from 1 to 32, at least one of the fatty chains is cis-unsaturated at one or more positions, and the fatty chains may be of different lengths and may be unsaturated at different locations. In some embodiments, when B is N, NR, or CR, then A is not H or OH when C₃ is —(CH₂)ₙ—.

The methods of the invention can be used to treat organelles, cells, tissues, organs and organ systems or components thereof. Examples of cells that can be treated include but are not limited to neurons, endothelial cells and cardiac muscle cells and organelles thereof. Examples of tissues that can be treated include but are not limited to epithelial tissue, muscle tissue, and nervous tissue. Examples of organs that can be treated include but are not limited to heart, blood vessels, spinal cord, and brain. Examples of organ systems that can be treated include but are not limited to the muscular system, the circulatory system, and the nervous system. Examples of components of organ systems that can be treated include but are not limited to the heart, the brain, and the blood vessels.

In the practice of the invention, PX-18 and other members of the PX compound family may be administered by any of many suitable methods known to those skilled in the art. If the veins are pretreated ex vivo prior to grafting, this may be accomplished, for example, by flushing the veins with a formulation that contains a PX compound such as PX-18. Once the veins are grafted into a patient (or prior to harvesting the veins from a donor), administration may be to the vein by methods known in the art, e.g. by intravenous or interarterial administration, or by systemic administration e.g. by injection, by oral administration, etc. Formulations of PX-18 and some related compounds are described, for example, in U.S. patent application Ser. No. 12/674,743 (filed Feb. 23, 2010, published as US2011/0097410), the complete contents of which are herein incorporated by reference. Other formulations of the PX family are described in U.S. Pat. Nos. 6,423,855; 6,020,489; 6,020,510; 5,859,271; and 5,659,055 (Franson et al.) the complete contents of which are herein incorporated by reference. Generally, the amount of PX-18 or PX-18 related compound in such a formulation will be in the range of from about 0.05-99%, w/v. The formulation may be, for example, a formulation of nanoparticles e.g. as suspensions or colloids.

The methods of the invention generally involve the administration of a therapeutically effective amount of a PX compound, e.g. PX-18. A “therapeutically effective amount” or a “therapeutic effective amount” is an amount that alleviates, totally or partially, the pathophysiological effects of shear stress on veins grafted during bypass surgery. The amount will depend upon, for example, patient size, gender, age, overall health, extent of the surgery being performed, and various genetic or non-genetic factors associated individual pharmacokinetic or pharmacodynamic properties of the administered compound. For a given subject in need thereof, a therapeutically effective amount can be determined by those of ordinary skill in the art and by methods known to those of ordinary skill in the art. That amount may be in the range of from about 1.0 to about 100 mg/kg introduced in vivo, or 0.01 to 20 mg/kg introduced in vitro or ex-vivo.

For post-graft administration, therapy may be acute or long-term. For example, for acute therapies, administration may begin immediately after transplant surgery and continue throughout the post-operative period (generally for several days, several weeks, or even for a few, e.g. 2-3, months). Alternatively, or in addition, therapy may be carried out or maintained on a long terms basis, e.g. for many months (e.g. 3-12 months), or several years, or even indefinitely during the remaining lifetime of the graft recipient.

In some embodiments the PX compounds, e.g. PX-18, are administered as liquids (e.g. suspensions or colloids) with a physiologically or pharmacologically suitable (compatible) carrier. The preparation of such compositions is well known to those skilled in the art. Typically, such compositions are prepared as colloidal suspensions. The active ingredients may be mixed with excipients which are pharmaceutically acceptable and compatible with the active ingredients. Suitable excipients are, for example, water, saline, dextrose, glycerol, ethanol and the like, or combinations thereof. In addition, the composition may contain minor amounts of auxiliary substances such as wetting or emulsifying agents, pH buffering agents, and the like. The final amount of active agent(s) in the formulations may vary. However, the amount of PX compound in such compositions will generally be in the range of about 0.01 to 10%. Further, more than one PX compound may be included in a formulation and administered to a patient. In particular, such formulations may include one or more surfactants and/or emulsifiers, examples of which include but are not limited to Tween® 80, Tween® 20 etc. In addition, the formulations may include one or more stabilizers, examples of which include but are not limited to Poloxamer 188, polyvinylpyrrolidone, egg lecithin, soy lecithin, etc. In one embodiment, the invention contemplates compositions particularly for intravenous (IV) administration of PX compounds, and the compositions of the invention may contain any additional ingredients required to provide the composition in a form suitable for stability and IV administration.

PX-18 and other PX compounds may also be administered in other oral and parenteral forms, which include but are not limited to, for example, various solid fauns suitable for suspension in liquids prior to administration, or solid forms that are administered without suspension. Examples of this aspect of the invention include various pills, powders, gels, pastes, ointments, etc. which may serve as a vehicle for delivery of PX nanoparticles. Those skilled in the art are familiar with the production of such drug vehicles and formulations, and all such that are known are intended to be encompassed by the present invention.

In a further embodiment of the invention, heparin or other anticoagulants are co-administered with the one or more PX-18 or PX-18 related compounds. Heparin in generally administered in an initial dose of about 250 IU/kg, to 400 IU/kg with supplemental heparin if activated coagulation time was greater than 480 or even 350 seconds, (levels that according to the published literature appears to result in acceptable fibrinopeptide A levels and post-cardiopulmonary bypass blood clotting, [38,39,40]), or appropriate dosages as determined by the attending medical professionals.

By “co-administration” we mean that the organelles, cells, tissues, organs and organ systems components thereof that are treated using the methods of the invention are exposed to at least two agents (anticoagulant and one or more compounds of the PX-18 family of compounds) at the same or at approximately the same time. For example, some formulations that are administered may contain both a PX-18 or PX-18 related compound and anticoagulant, e.g. in a single mixture formulated for intravenous administration, or for treating a vein ex vivo, etc., so that administration is simultaneous. Alternatively, these agents may be administered one at a time, one after the other, with either being administered first, but within a time period so that for at least part of the treatment period, the treated organelles, cells, tissues, organs and organ systems or components thereof are exposed to both agents.

In some embodiments of the invention the patient or subject is a mammal, frequently a primate, and in preferred embodiments of the invention the mammal is a human, although this need not be the case. Veterinary applications of this technology are also contemplated.

The invention is further illustrated in the following examples, which should not be construed as limiting in any way.

### EXAMPLES

As discussed in the Background section, the multivessel nature of coronary artery disease and ready availability of saphenous veins results in their use in over 70% of CABG procedures, despite the superiority of arterial graft patency over that of vein grafts. Difficulties associated with the use of vein grafts is likely due to injury due to surgical preparation, and the increased pressure on the venous grafts, which distends the arterial system, can eventually also cause pathological changes, such as mural thinning and endothelial damage.

It is known that the expression of the enzyme sPLA₂-IIA in vascular smooth muscle cells plays an essential role in the progression of atherosclerotic plaques [14]. It is possible that serum sPLA₂-IIA also promote endothelial dysfunction by its ability to promote generation of lysophosphatidylcholine, a known inhibitor of eNOS [13]. This enzyme is thus a candidate for a causative agent of vein graft damage, and the present studies investigated, for the first time, an in vitro mechanical stretch model of endothelial cells and the possible role of sPLA₂-IIA in vein graft damage.

### Example 1

### The Effect of PX-18 in an In-Vitro Model of Human Perfused Veins

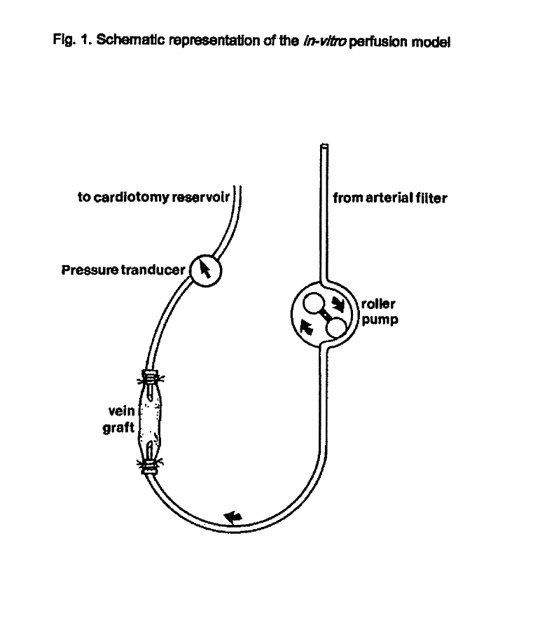

Veins were surplus segments of harvested saphenous veins of patients who underwent CABG. Vein specimens were collected in the operating room under sterile conditions for histopathological examination. The veins were perfused with autologous blood (n=6: control), or perfused with autologous blood supplemented with PX-18, a specific sPLA2-IIA inhibitor (diluted to 0.5 mg/ml of blood supply, which is equivalent to an in vivo dosage of approximately 30 mg/kg) in an experimental set-up utilizing a small roller pump and a vein irrigation set, described in FIG. 1. Perfusion pressure was about 60 mm Hg by adjusting the flow. At no point in the harvesting procedure was distention allowed before perfusion in the perfusion system. Flow in the vein grafts was about 100 ml/min. The veins were analyzed after 6 hours of perfusion.

Paraffin embedded vein sections (4 μm thick) were mounted on microscope slides and deparaffinized for 10 minutes in xylene at room temperature and dehydrated through descending concentrations of ethanol. Endogenous peroxidase activity was blocked by incubation in 0.3% (v/v) H₂O₂ in methanol for 30 minutes. Tissue sections were subjected to antigen retrieval by boiling in 10 mM sodium citrate buffer, pH 6, for 10 minutes in a microwave oven. Antibody and normal serum were diluted in Phosphate Buffered Saline (PBS) containing 1% (w/v) bovine serum albumin (BSA). Tissue sections were pre-incubated for 10 mM with normal rabbit serum, followed by incubation for 1 hour with 5 μg/ml monoclonal antibody 4A1 against human sPLA₂-IIA and monoclonal human antibody CD34, to detect endothelial cells, in PBS Bovine Serum Albumin 1% for 60 minutes at room temperature.

After washing in PBS, sections were incubated for 30 minutes with a biotin-conjugated secondary antibody (rabbit anti-mouse biotin 1:500 dilution). After again washing in PBS, slides were incubated with streptavidin-biotin complex for 1 hour and were visualized with 3,3′-diaminobenzidine (DAB); 0.1 mg/ml, 0.02% H₂O₂). Slides were counterstained with haematoxylin and mounted with Depex.

The percentage of CD34 positive endothelium in each section was quantified by computer-assisted morphometry. One to three sections of the vein from each patient were analyzed. Only sections where the total circumference of the vein was visible included in the analysis. An average score for all analyzed sections in each group (with and without PX-18), was defined as the percentage of CD34 positive area.

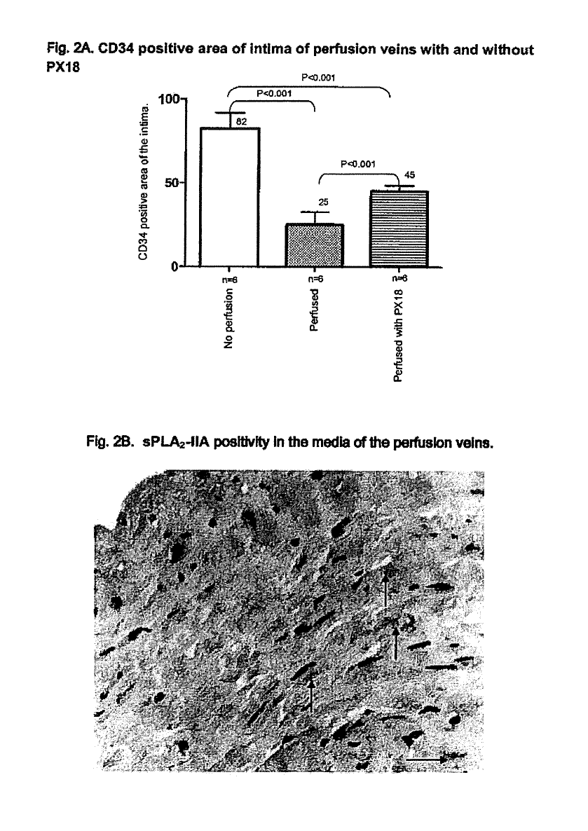

Veins were perfused for six hours in 2 different perfusion systems, one with and one without PX-18. In freshly harvested veins, prior to perfusion, an 82.4%±9.5% CD34 positive endothelial cell area was found. After perfusion with blood without PX-18, the percentage of CD34 positive endothelial cell area was reduced to 24.8%±7.7% (p<0.001). After perfusion with blood supplemented with PX-18, the CD34 positive endothelial cell area declined to 45.0±8.2% (p<0.001) PX-18 (FIG. 2A), an improvement in endothelial cell coverage area of 81%

In perfusion veins, focal weak sPLA₂-IIA was found within the extracellular matrix and in the smooth muscle cells of the longitudinal and circular layers of the media, but not in (residual) endothelial cells. The amount of sPLA₂-IIA positive cells, however did not differ significantly between veins perfused with or without PX-18.

### Example 2

### Effect of Endothelial Cell Stretching

Venous grafts within arterial systems are distended by pressures that cause increased circumferential and radial stresses. This mechanical stretch is associated with endothelial damage [20]. The effect of PX-18 on stretch-induced endothelial damage in vitro was investigated. For this the level of caspase-3 activity was measured, as a marker for apoptosis in HUVECs subjected to 24 hour stretching, in the presence or absence of PX-18.

Human umbilical vein endothelial cells (HUVECs) were isolated from umbilical cords as described in [18] and cultured in Medium 199, supplemented with 10% heat-inactivated fetal calf serum, 10% heat-inactivated human serum, 5 units/ml heparin, 50 mg/ml endothelial cell growth factor, penicillin and streptomycin (at 37□C in 5% CO₂/95% air atmosphere at a relative humidity of 50%. Experiments were performed at 100% confluence of the cells and were used at passages 2 and 3. HUVECs were seeded in 6-well plates, coated with collagen I-coated flexible membranes and fibronectin. The cells were cultured with and without PX-18, diluted in normal culture medium (final concentration of 0.2 mg/ml). Subsequently, the cells were stretched in the Flexercell FX4000 apparatus at 10% stretch at 1 Hz for 24 hours.

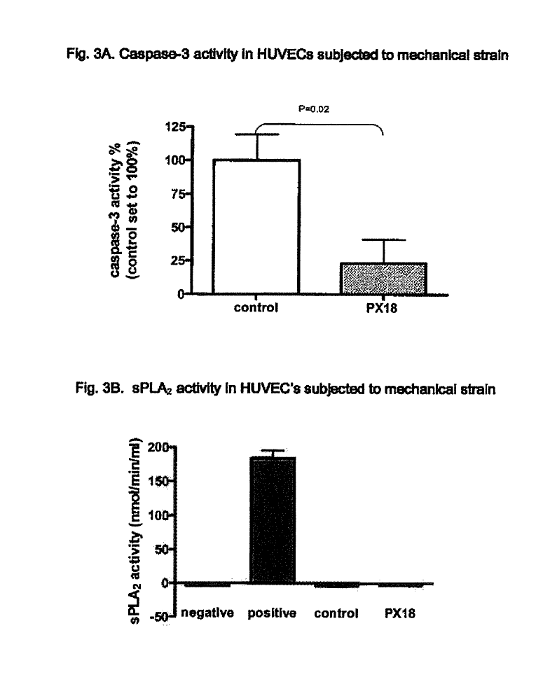

After 24 hours stretching, the HUVECs were counted and transferred to a 96-well plate (30,000 cells/well). The cells were lysed and incubated with DEVD-rhodamine 110 substrate for one hour at 37° C. Subsequently, the fluorescence of free rhodamine was determined with a microplate fluorescence reader. The developed fluorochrome was proportional to the concentration of activated caspase-3 and was quantified by a calibration curve of diluted free rhodamine. It was found that there was a 76% decrease of caspase-3 activity in stretched HUVECs in the presence of PX-18, compared to stretched HUVECs in the absence of PX-18 (FIG. 3A; p=0.02).

### Example 3

The role of sPLA₂-IIA in apoptosis of stretched HUVECs is evaluated. Since stretch of HUVECs was performed in the absence of sPLA₂-IIA, the question of whether HUVECs synthesized was also analyzed. Whole cell lysates were analyzed for the presence of sPLA₂ activity. In brief, 10 μl of cell lysate was added to an assay mix containing the substrate diheptanoyl phosphatidylcholine, and 5,5-dithio-bis-2-nitrobenzoic acid (DTNB). As a negative control the substrate containing assay mix without added lysate was used. As a positive control, purified type III sPLA₂ (bee venom sPLA₂) was used. sPLA₂ activity was determined by measuring the release of free thiols in a microplate fluorescence reader. No sPLA₂-IIA activity was detected in HUVEC's, stretched either in the presence or the absence of PX-18. In both cases sPLA₂-IIA values were identical to the negative control (FIG. 3B). These findings indicate that PX-18 protects HUVECs from stretch-induced apoptosis in a manner independent of its inhibition of sPLA₂-IIA.

### Discussion:

Although there is a considerable amount of data demonstrating sPLA₂-IIA expression in arteries [24; 25], not much is known about sPLA₂-IIA expression in veins, apart from Ost et al's observation of sPLA₂-IIA mRNA expression in human umbilical venous biopsies [26]. In the example presented here, only weak expression of sPLA₂-IIA in perfused saphenous veins was found, and then only in the media, not in residual endothelial cells. Furthermore, PX-18 did not have an effect on this expression of sPLA₂-IIA in the residual endothelial cells. Nor could sPLA₂-IIA activity be detected in stretched HUVEC's. This evidence indicates that PX-18 acts to inhibit apoptosis in vein grafts (both stretch-induced and other) in a manner independently from its ability to inhibit sPLA₂-IIA. However, this evidence does not exclude the possibility that part of the in vivo protection offered by PX-18 results from its inhibitory effect on circulating sPLA₂-IIA. Indeed it is known that sPLA₂-IIA serum levels are increased in patients in the first days after undergoing CABG comparable to those of C reactive protein (CRP), independent of pump use for extracorporeal circulation during the procedure [27; 28]. In addition, heparin, routinely given to patients who underwent CABG, was found to induce increased circulating sPLA₂-IIA levels [29; 30].

The role of sPLA₂-IIA in atherosclerosis development and progression in arteries has been well described [34]. For example, evidence has been found that sPLA₂-IIA is involved in the development of plaque instability in coronary arteries leading to plaque complications such as acute myocardial infarction [16]. It has been reported that 1-H-indole-3-glyoxamide, another inhibitor of sPLA₂-IIA enzymes, can reduce atherosclerosis in mice, guinea pigs and humans [35-37]. It has also been noted that early endothelial damage in vein grafts may initiate the development of atherosclerosis by stimulating migration and proliferation of smooth muscle cells into the intima [21]. It is therefore concluded that limiting early postoperative endothelial damage in vein grafts should limit the rate and extent of graft atherosclerosis development.

The inhibitory effect of PX-18 and similar compounds on circulating sPLA₂-IIA may also inhibit graft atherosclerosis development. The role of sPLA₂-IIA in atherosclerosis development and progression in arteries has been well described [30]. There is also evidence that sPLA2-IIA is involved in the development of plaque instability in coronary arteries leading to plaque complications such as acute myocardial infarction [15]. Vein grafts develop atherosclerosis at a faster rate, on average, than nearby arterial walls. Atherosclerotic changes have been reported as early as two to three months postoperatively [31], the incidence of which increases in time [32), and virtually all saphenous vein grafts older than one year show atherosclerotic plaque formation [33]. Thus, the limiting of early postoperative endothelial damage in vein grafts through the use of PX-18 is expected to slow the rate and extent of graft atherosclerosis development. Other PX compounds may have similar protective properties.

### REFERENCES

1. Rueda F, Souza D, Lima Rde C, Menezes A, Johansson B, Dashwood M, Thé E, Gesteira M, Escobar M, Vasconcelos F. Novel no-touch technique of harvesting the saphenous vein for coronary artery bypass grafting. Arq Bras Cardiol. 2008 June; 90(6):356-62. English, Portuguese.
2. Motwani J G, Topol E J. Aortocoronary saphenous vein graft disease. Pathogenesis, predisposition and prevention. Circulation. 1998; 97:916-931.
3. Angelini G D, Newby A C. The future of saphenous vein as a coronary artery bypass conduit. Eur Heart J. 1989; 10:273-280
4. J. H. Kennedy, M. J. Lever, B. J. Addis and M. Paneth, Changes in vein interstitium following distension for aortocoronary bypass. J. Cardiovasc. Surg. (Torino) 30 (1989), pp. 992-995.
5. Lau G T, Ridley L J, Bannon P G, Wong L A, Trieu J, Brieger D B, Lowe H C, Freedman B S, Kritharides Lumen loss in the first year in saphenous vein grafts is predominantly a result of negative remodeling of the whole vessel rather than a result of changes in wall thickness. Circulation. 2006 Jul. 4; 114(1 Suppl):1435-40.
6. Hata M, Sezai A, Niino T, Yoda M, Wakui S, Chiku M, Takayama T, Honye J, Saitoh S, Minami K. What is the optimal management for preventing saphenous vein graft diseases? early results of intravascular angioscopic assessment. Circ J. 2007 February; 71(2):286-7.
7. Fischell T A, Subraya R G, Ashraf K, Perry B, Haller S. Pharmacologic” distal protection using prophylactic, intragraft nicardipine to prevent no-reflow and non-Q-wave myocardial infarction during elective saphenous vein graft intervention. J Invasive Cardiol. 2007 February; 19(2):58-62.
8. Cacho F, Doblare M, Holzapfel G A. A procedure to simulate coronary artery bypass graft surgery. Med Biol Eng Comput. 2007 September; 45(9):819-27. Epub 2007 Aug. 3.
9. Kockx M M, Cambier B A, Bortier H E, De Meyer G R, Van Cauwelaert P A. The modulation of smooth muscle cell phenotype is an early event in human aorto-coronary saphenous vein grafts. Virchows Arch A Pathol Anat Histopathol. 1992; 420(2):155-62.
10. Sattar N. Inflammation and endothelial dysfunction: intimate companions in the pathogenesis of vascular disease? Clin Sci (Lond). 2004 May; 106 (5):443-5.
11. Vaudo, G., Marchesi, S., Gerli, R. et al. Endothelial dysfunction in young patients with rheumatoid arthritis and low disease activity. Ann. Rheum. Dis. 2004; 63, 31-35
12. Sattar, N., McCarey, D. W., Capell, H. and McInnes, I. B. Explaining how ‘high-grade’ systemic inflammation accelerates vascular risk in rheumatoid arthritis. Circulation 2003; 108, 2957-2963
13. Fichtlscherer, S., Kaszkin, M., Breuer, S., Dimmeler, S, and Zeiher, A. M. Elevated secretory non-pancreatic type II phospholipase A₂ serum activity is associated with impaired endothelial vasodilator function in patients with coronary artery disease. Clin. Sci. 2004; 106, 511-517
14. Jaulmes A, Thierry S, Janvier B, Raymondjean M and Maréchal V, Activation of sPLA2-IIA and PGE2 production by high mobility group protein B1 in vascular smooth muscle cells sensitized by IL-1β. The FASEB Journal. 2006; 20:1727-1729.
15. Nijmeijer R, Meuwissen M, Krijnen P A, van der Wal A, Piek J J, Visser C A, Hack C E, Niessen H W. Secretory type II phospholipase A2 in culprit coronary lesions is associated with myocardial infarction. Eur J Clin Invest. 2008; 38:205-10.
16. van Dijk A, Krijnen P A, Vermond R A, Pronk A, Spreeuwenberg M, Visser F C, Berney R, Paulus W J, Hack C E, van Milligen F J, Niessen H W. Inhibition of type 2A secretory phospholipase A2 reduces death of cardiomyocytes in acute myocardial infarction. Apoptosis. 2009 June; 14:753-63.
17. W Stooker, H W M Niessen, A Baidoshvili, W R Wildevuur, V W M Van Hinsbergh, J Fritz, Ch R H Wildevuur and L Eijsman, Perivenous support reduces early changes in human vein grafts: studies in whole blood perfused human vein segments. J Thorac Cardiovasc Surg 2001; 121; 290-297.
18. Jaffe E A, Nachman R L, Becker C G and Minick C R. Culture of human endothelial cells derived from umbilical veins. Identificacion by morphologic and immunologic criteria. J Clin Invest 1973; 52: 2745-2756.
19. Volanakis J E, Narkates A J (1981) Interaction of C-reactive protein with artificial phosphatidylcholine bilayers and complement. J Immunol 126:1820-1825
20. Stooker W, Niessen H W, Wildevuur W R, van Hinsbergh V W, Fritz J, Jansen E K, Wildevuur ChR, Eijsman L. Perivenous application of fibrin glue reduces early injury to the human saphenous vein graft wall in an ex vivo model. Eur J Cardiothorac Surg. 2002 February; 21(2): 212-7.
21. Raja S G, Haider Z, Ahmad M, Zaman H. Saphenous vein grafts: to use or not to use? Heart Lung Circ. 2004; 13:403-9.
22. J. H. Kennedy, M. J. Lever, B. J. Addis and M. Paneth, Changes in vein interstitium following distension for aortocoronary bypass. J. Cardiovasc. Surg. (Torino) (1989); 30; 992-995.
23. Stooker W, Niessen H W, Jansen E K, Fritz J, Wildevuur W R, Van Hinsbergh V W, Wildevuur ChR, Eijsman L. Surgical sealant in the prevention of early vein graft injury in an ex vivo model. Cardiovasc Pathol. 2003; 12: 202-6.
24. Jaross W, Eckey R, Menschikowski M: Biological effects of secretory phospholipase A(2) group IIA on lipoproteins and in atherogenesis. Eur J Clin Invest, 2002; 32: 383-93
25. Hurt-Camejo E, Andersen S, Standal R, Rosengren B, Sartipy P, Stadberg E, Johansen B. Localization of nonpancreatic secretory phospholipase A2 in normal and atherosclerotic arteries. Activity of the isolated enzyme on low-density lipoproteins. Arterioscler Thromb Vasc Biol. 1997 February; 17(2):300-9.
26. Ost M, Uhl E, Carlsson M, Gidlöf A, Söderkvist P, Sirsjö A. Expression of mRNA for phospholipase A2, cyclooxygenases, and lipoxygenases in cultured human umbilical vascular endothelial and smooth muscle cells and in biopsies from umbilical arteries and veins. J Vasc Res. 1998 May-June; 35:150-5.
27. Grönroos J M, Kuttila K, Perttilä J, Nevalainen T J. Phospholipases A2 in the serum of patients after coronary artery bypass surgery. Crit. Care Med. 1996 February; 24:259-62.
28. Varnous S, Ben Ayed S, Masliah J, Leprince P, Gandjbakhch I, Bernard M, Beaudeux J L. Secretory phospholipase A(2) activity and release kinetics of vascular tissue remodelling biomarkers after coronary artery bypass grafting with and without cardiopulmonary bypass. Clin Chem Lab Med. 2007; 45:372-5.
29. Nakamura H, Kim D K, Philbin D M, Peterson M B, Debros F, Koski G, Bonventre J V. Heparin-enhanced plasma phospholipase A2 activity and prostacyclin synthesis in patients undergoing cardiac surgery. J Clin Invest. 1995; 95:1062-70.
30. Kern H, Johnen W, Braun J, Frey B, Rastow B, Kox W J, Schlame M. Heparin induces release of phospholipase A(2) into the splanchnic circulation. Anesth Analg. 2000; 91: 528-32.
31. Grondin C M. Graft disease in patients with coronary bypass grafting. Why does it start? Where do we stop? J Thorac Cardiovasc Surg. 1986 September; 92:323-329.
32. Fuchs J C, Mitchener J S 3rd, Hagen P O. Postoperative changes in autologous vein grafts. Ann Surg. 1978; 188:1-15.
33. Kalan J M, Roberts W C. Morphologic findings in saphenous veins used as coronary arterial bypass conduits for longer than 1 year: necropsy analysis of 53 patients, 123 saphenous veins, and 1865 five-millimeter segments of veins. Am Heart J. 1990; 119: 1164-84.
34. Krijnen P A. J. Nijmeijer R, Hack C E, Niessen H W M. Group II Secretory PLA2: A New Cardiovascular Risk Factor. Anti-Inflammatory & Anti-Allergy Agents in Medicinal Chemistry. 2006; 5; 163-173.
35. Fraser H, Hislop C, Christie R M, Rick H L, Reidy C A, Chouinard M L, Eacho P I, Gould K E, Trias J. Varespladib (A-002), a Secretory Phospholipase A2 Inhibitor, Reduces Atherosclerosis and Aneurysm Formation in ApoE−/− Mice. J Cardiovasc Pharmacol. 2009
36. Leite J O, Vaishnav U, Puglisi M, Fraser H, Trias J, Fernandez M L. A-002 (Varespladib), a phospholipase A2 inhibitor, reduces atherosclerosis in guinea pigs. BMC Cardiovasc Disord. 2009 Feb. 17; 9:7.
37. Rosenson R S, Hislop C, McConnell D, Elliott M, Stasiv Y, Wang N, Waters D D; PLASMA Investigators. Effects of 1-H-indole-3-glyoxamide (A-002) on concentration of secretory phospholipase A2 (PLASMA study): a phase II double-blind, randomised, placebo-controlled trial. Lancet. 2009 Feb. 21; 373:649-58.
38. Despotis G J, Joist J H, Hogue C W Jr, Alsoufiev A, Kater K, Goodnough L T, Santoro S A, Spitznagel E, Rosenblum M, Lappas D G. The impact of heparin concentration and activated clotting time monitoring on blood conservation. A prospective, randomized evaluation in patients undergoing cardiac operation. J Thorac Cardiovasc Surg. 1995 July; 110(1):46-54.
39. Gravlee G P, Haddon W S, Rothberger H K, Mills S A, Rogers A T, Bean V E, Buss D H, Prough D S, Cordell A R. Heparin dosing and monitoring for cardiopulmonary bypass. A comparison of techniques with measurement of subclinical plasma coagulation. J Thorac Cardiovasc Surg. 1990 March; 99(3):518-27.
40. Metz S, Keats A S. Low activated coagulation time during cardiopulmonary bypass does not increase postoperative bleeding. Ann Thorac Surg. 1990 March; 49(3):440-4.

I claim:

1. A method of protecting organelles, cells, tissues, organs and organ systems or components thereof from the deleterious effects of mechanical damage and subsequent biochemical damage stemming from said mechanical damage, said method comprising the step of treating said organelles, cells, tissues, organs and organ systems or components thereof with one or more PX-18 compounds.

2. The method of claim 1, wherein said mechanical damage is caused by stretching and sheer stress.

3. The method of claim 1, whereas said component of an organ system is a vein used to replace a portion of an artery in vein-graft, artery bypass surgery.

4. A method of claim 3, wherein said treating step limits endothelial damage in said vein and reduces the build-up of arteriosclerotic plaque in said vein.

5. The method of claim 1, wherein said treating step includes the step of administering said one or more PX-18 or PX-18 related compounds to a donor in vivo before said vein, organelles, cells, tissues, organs, or organ systems or components thereof are harvested from said donor.

6. The method of claim 3, wherein said treating step includes the step of administering said one or more PX-18 or PX-18 related compounds to a donor in vivo before said vein, organelles, cells, tissues, organs, or organ systems or components thereof are harvested from said donor.

7. The method of claim 1, wherein said method is carried out ex vivo after said vein, organelles, cells, tissues, organs, or organ systems or components thereof are harvested from a donor.

8. The method of claim 1, wherein said method is carried out ex vivo after said vein, organelles, cells, tissues, organs, or organ systems or components thereof are harvested from a donor.

9. The method of claim 1, wherein said treating step includes the step of administering said one or more PX-18 or PX-18 related compounds to a recipient in vivo before, after, or both before and after said vein, organelles, cells, tissues, organs, and organ systems or components thereof are grafted into said recipient.

10. The method of claim 9, wherein said step of administering is carried out during an acute post-operative period.

11. The method of claim 9, wherein said step of administering is carried out as long-term maintenance therapy.

12. The method of claim 3, wherein said treating step includes the step of administering said one or more PX-18 or PX-18 related compounds to a recipient in vivo before, after, or both before and after said vein, organelles, cells, tissues, organs, and organ systems or components thereof are grafted into said recipient.

13. The method of claim 12, wherein said step of administering is carried out during an acute post-operative period.

14. The method of claim 12, wherein said step of administering is carried out as long-teen maintenance therapy.

15. The method of claim 1, wherein said one or more PX compounds includes PX-18.

16. The method of claim 3, wherein said one or more PX compounds includes PX-18.

17. A method of limiting endothelial damage in vein grafts and reducing the build-up of arteriosclerotic plaque in said vein grafts comprising, providing one or more anticoagulants and one or more PX-18 compounds to endothelium of said vein graft.

18. A method of claim 17 whereas said anticoagulant is heparin.

19. The method of claim 17, wherein said one or more PX-18 compounds includes PX-18.
